(19) United States
(12) Patent Application Publication
(10) Pub. No.: US 2009/0163719 A1
(43) Pub. Date: Jun. 25, 2009

(54) CATALYST COMPOSITIONS AND THEIR USE IN THE DE-ENRICHMENT OF ENANTIOMERICALLY ENRICHED SUBSTRATES

(76) Inventors: Andrew John Blacker, West Yorkshire (GB); Matthew John Stirling, West Yorkshire (GB)

Correspondence Address: MYERS BIGEL SIBLEY & SAJOVEC, PO BOX 37428, RALEIGH, NC, 27627, US

(21) Appl. No.: 11/577,912

(22) PCT Filed: Oct. 27, 2005
(86) PCT No.: PCT/GB05/04176

§ 371 Date: Feb. 18, 2009

(30) Foreign Application Priority Data
Oct. 29, 2004 (GB) 0424002.4
Apr. 20, 2005 (GB) 0507960.3

(51) Int. Cl. C07D 217/02; C07C 211/00; C07D 207/10

(52) U.S. Cl. 546/150; 564/387; 564/384; 548/532

(57) ABSTRACT

There is provided a process for the de-enrichment of enantiomerically enriched compositions which comprises reacting an enantiomerically enriched composition comprising at least a first enantiomer or diastereomer of a substrate comprising a carbon-heteroatom bond, wherein the carbon is a chiral centre and the heteroatom is a group V heteroatom, in the presence of a catalyst system and optionally a reaction promoter to give a product composition comprising first and second enantiomers or diastereomers of the substrate having a carbon-heteroatom bond, the ratio of second to first enantiomer or diastereomer in the product composition being greater than the ratio of second to first enantiomer or diastereomer in the enantiomerically enriched composition. Preferred catalyst systems include transition metal halide complex of the formula MₙXₚYᵣ wherein M is a transition metal; X is a halide; Y is a neutral optionally substituted hydrocarbyl complexing group, a neutral optionally substituted perhalogenated hydrocarbyl complexing group, or an optionally substituted cyclopentadienyl complexing group; and n, p and r are integers. The reaction promoter is preferably a halide salt.

The invention concerns a process for the de-enrichment of enantiomerically enriched substrates, especially amines.

According to a first aspect of the present invention, there is provided a process for the de-enrichment of enantiomerically enriched compositions which comprises reacting an enantiomerically enriched composition comprising at least a first enantiomer or diastereomer of a substrate comprising a carbon-heteroatom bond, wherein the carbon is a chiral centre and the heteroatom is a group V heteroatom, in the presence of a catalyst system and optionally a reaction promoter to give a product composition comprising first and second enantiomers or diastereomers of the substrate having a carbon-heteroatom bond, the ratio of second to first enantiomer or diastereomer in the product composition being greater than the ratio of second to first enantiomer or diastereomer in the enantiomerically enriched composition.

Preferably the product composition is a racemic mixture of the first and second enantiomers of the substrate comprising a carbon-heteroatom bond, wherein the carbon is a chiral centre.

Substrates which may be enantiomerically de-enriched by the process of the present invention include amines at a chiral secondary carbon atom and ammonium salts chiral at a secondary carbon atom.

Preferably, in the process of the present invention, the substrate comprising a carbon-heteroatom bond, the carbon atom being a chiral centre, is a compound of formula (1):

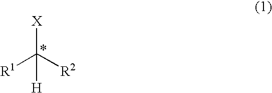

wherein:
X represents NHR³, NR³R⁴, (NHR³R⁴)⁺Q⁻;
Q⁻ represents an anion;
R¹, R² each independently represents an optionally substituted hydrocarbyl, a perhalogenated hydrocarbyl, an optionally substituted heterocyclyl group or a substitutent group;
R³ represents a hydrogen atom, an optionally substituted hydrocarbyl, a perhalogenated hydrocarbyl, an optionally substituted heterocyclyl group or a removable group;
R⁴ represents a hydrogen atom, an optionally substituted hydrocarbyl, a perhalogenated hydrocarbyl or an optionally substituted heterocyclyl group; or one or more of R¹ & R², R¹ & R³, R² & R⁴ and R³ & R⁴ optionally being linked in such a way as to form an optionally substituted ring(s);
   provided that R¹, R², R³ and R⁴ are selected such that * is a chiral centre.

Hydrocarbyl groups which may be represented by R¹⁻⁴ independently include alkyl, alkenyl and aryl groups, and any combination thereof, such as aralkyl and alkaryl, for example benzyl groups.

Alkyl groups which may be represented by R¹¹ include linear and branched alkyl groups comprising up to 20 carbon atoms, particularly from 1 to 7 carbon atoms and preferably from 1 to 5 carbon atoms. When the alkyl groups are branched, the groups often comprising up to 10 branch chain carbon atoms, preferably up to 4 branch chain atoms. In certain embodiments, the alkyl group may be cyclic, commonly comprising from 3 to 10 carbon atoms in the largest ring and optionally featuring one or more bridging rings. Examples of alkyl groups which may be represented by R¹⁻⁴ include methyl, ethyl, propyl, 2-propyl, butyl, 2-butyl, t-butyl and cyclohexyl groups.

Alkenyl groups which may be represented by R¹⁻⁴ include C₂₋₂₀, and preferably C₂₋₆ alkenyl groups. One or more carbon-carbon double bonds may be present. The alkenyl group may carry one or more substituents, particularly phenyl substituents. Examples of alkenyl groups include vinyl, styryl and indenyl groups. When either of R¹ or R² represents an alkenyl group, a carbon-carbon double bond is preferably located at the position p to the C-heteroatom moiety.

Aryl groups which may be represented by R¹⁻⁴ may contain 1 ring or 2 or more fused rings which may include cycloalkyl, aryl or heterocyclic rings. Examples of aryl groups which may be represented by R¹⁻⁴ include phenyl, tolyl, fluorophenyl, chlorophenyl, bromophenyl, trifluoromethylphenyl, anisyl, naphthyl and ferrocenyl groups.

Perhalogenated hydrocarbyl groups which may be represented by R¹⁻⁴ independently include perhalogenated alkyl and aryl groups, and any combination thereof, such as aralkyl and alkaryl groups. Examples of perhalogenated alkyl groups which may be represented by R¹⁻⁴ include —CF₃ and —C₂F₅.

Heterocyclic groups which may be represented by R¹⁻⁴ independently include aromatic, saturated and partially unsaturated ring systems and may constitute 1 ring or 2 or more fused rings which may include cycloalkyl, aryl or heterocyclic rings. The heterocyclic group will contain at least one heterocyclic ring, the largest of which will commonly comprise from 3 to 7 ring atoms in which at least one atom is carbon and at least one atom is any of N, O, S or P. When either of R¹ or R² represents or comprises a heterocyclic group, the atom in R¹ or R² bonded to the C-heteroatom group is preferably a carbon atom. Examples of heterocyclic groups which may be represented by R¹⁻⁴ include pyridyl, pyrimidyl, pyrrolyl, thiophenyl, furanyl, indolyl, quinolyl, isoquinolyl, imidazoyl and triazoyl groups.

Removable groups which may be represented by R³ include —P(O)R⁵R⁶, —P(O)OR⁷OR⁸, —P(O)OR⁷OH, —P(O)(OH)₂, —P(O)SR⁹SR¹⁰, —P(O)SR⁹SH, —P(O)(SH)₂, —P(O)NR¹¹R¹²NR¹³R¹⁴—P(O)NR¹¹R¹²NHR¹³—P(O)NHR¹¹NHR¹³, —P(O)NR¹¹R¹²NH₂, —P(O)NHR¹¹NH₂, —P(O)(NH₂)₂, —P(O)R⁵OR⁷, —P(O)R⁶OH, —P(O)R⁵SR⁹, —P(O)R⁵SH, —P(O)R⁵NR¹¹R¹², —P(O)R⁵NHR¹¹, —P(O)R⁵NH₂, —P(O)OR⁷SR⁹, —P(O)OR⁷SH, —P(O)OHSR⁹, —P(O)OHSH, —P(O)OR⁷NR¹¹R¹², —P(O)OR⁷NHR¹¹, —P(O)OR⁷NH₂, —P(O)OHNR¹¹R¹²—P(O)OHNHR¹¹, —P(O)OHNH₂, —P(D)SR⁹NR¹¹R¹², —P(O)SR⁹NHR¹¹, —P(O)SR⁹NH₂, —P(O)SHNR¹¹R¹², —P(O)SHNHR¹¹, —P(O)SHNH₂, —P(S)R⁵R⁶, —P(S)OR⁷OR⁸, —P(S)OR⁷OH, —P(S)(OH)₂, —P(S)SR⁹SR¹⁰, —P(S)SR⁹SH, —P(S)(SH)₂, —P(S)NR¹¹R¹²NR¹³R¹⁴, —P(S)NR¹¹R¹²NHR¹³, —P(S)NHR¹¹NHR¹³, —P(S)NR¹¹R¹²NH₂, —P(S)NHR¹¹NH₂, —P(S)(NH₂)₂, —P(S)R⁵OR⁷, —P(S)R⁵OH, —P(S)R⁵SR⁹, —P(S)R⁵SH, —P(S)R⁵NR¹¹R¹², —P(S)R⁵NHR¹¹, —P(S)R⁵NH₂, —P(S)OR⁷SR⁹, —P(S)OHSR⁹, —P(S)OR⁷SH, —P(S)OHSH, —P(S)OR⁷NR¹¹R¹²—P(S)OR⁷NHR¹¹, —P(S)OR⁷NH₂, —P(S)OHNR¹¹R¹², —P(S)OHNHR¹¹, —P(S)OHNH₂, —P(S)SR⁹NR¹¹R¹², —P(S)SR⁹NHR¹¹, —P(S)SR⁹NH₂, —P(S)SHNR¹¹R¹², —P(S)SHNHR¹¹, —P(S)SHNH₂, —PR⁵R⁶, —POR⁷OR⁸, —PSR⁹SR¹⁰, —PNR¹¹R¹²NR¹³R¹⁴, —PR⁵OR⁷PR⁵SR⁹, —PR⁶NR¹¹R¹², —POR⁷SR⁹, —POR⁷NR¹¹R¹², —PSR⁹NR¹¹R¹², —S(O)R¹⁵, —S(O)₂R¹⁶, —COR¹⁷, —CO₂R¹⁸, —SiR¹⁹R²⁰R²¹, —OH, —OR²², —OC(O)R²³, NR²⁶R²⁷ or N═CR²⁸R²⁹ wherein R⁵ and R⁶ independently represent an optionally substituted hydrocarbyl, a perhalogenated hydrocarbyl, an optionally substituted heterocyclyl group or —N═CR²⁴R²⁵ where R²⁴ and R²⁵ are as defined for R¹; R⁷ to R²³ each independently represents an optionally substituted hydrocarbyl, a perhalogenated hydrocarbyl or an optionally substituted heterocyclyl group, and R²⁵ to R²⁹ each independently represents hydrogen, an optionally substituted hydrocarbyl, a perhalogenated hydrocarbyl or an optionally substituted heterocyclyl group.

When any of R¹⁻²⁹ is a substituted hydrocarbyl or heterocyclic group, the substituent(s) should be such so as not to adversely affect the rate or stereoselectivety of the reaction. Optional substituents include halogen, cyano, nitro, hydroxy, amino, thiol, acyl, hydrocarbyl, perhalogentated hydrocarbyl, heterocyclyl, hydrocarbyloxy, mono or di-hydrocarbylamino, hydrocarbylthio, esters, carbonates, amides, sulphonyl and sulphonamido groups wherein the hydrocarbyl groups are as defined for R¹ above. One or more substituents may be present.

Substituent groups which may be represented by R¹ or R² include halogen, cyano, nitro, hydroxy, amino, thiol, acyl, carboxyl, hydrocarbyloxy, mono or di-hydrocarbylamino, hydrocarbylthio, esters, carbonates and amides groups.

When any of R¹ & R², R¹ & R³, R² & R⁴, and R³ & R⁴ are linked in such a way that when taken together with either the carbon atom and/or atom X of the compound of formula (1) that a ring is formed, it is preferred that these be 5, 6 or 7 membered rings and optionally containing one or more ring heteroatoms, preferably O, S or N ring atoms. Examples of such compounds of formula (1) include 1-methyl-1,2,3,4-tetrahydroisoquinoline, 1-phenyl-1,2,3,4-tetrahydroisoquinoline and 1-methyl-6,7-dimethoxy-1,2,3,4-tetrahydroisoquinoline.

Compounds of formula (1) where X is represented by NHR³, NR³R⁴, (NHR³R⁴)⁺Q⁻, include amines or ammonium salts. Where a compound of formula (1) is an amine, it may optionally be converted to an ammonium salt. Preferred ammonium salts are represented by compounds of formula (1) in which X is (NHR³R⁴)⁺Q⁻ wherein R³ or R⁴ are the same or different. When the compound of formula (1) is an ammonium salt, an anion represented by Q⁻ is present. Examples of anions which may be present are halide, hydrogen sulphate, carbonate, hydrogencarbonate, tosylate, formate, acetate tetrafluoroborate, trifluoromethanesulphonate and trifluoroacetate.

In certain preferred embodiments, R¹ and R² are both different and selected to both be different C₁₋₆ alkyl groups, both be different aryl groups, particularly where one is a phenyl group, or are selected such that one is aryl, particularly phenyl and one is C₁₋₆ alkyl. Substituents may be present, particularly substituents para to the C—X group when one or both of R¹ and R² is a substituted phenyl group.

Examples of compounds of formula (1) include N-methyl-1-phenylethylamine, N-benzyl-1-phenylethylamine, 1-(2-naphthyl)ethylamine, 1-(1-naphthyl)ethylamine and 1-phenylethylamine.

The catalyst system preferably comprises a transition metal catalyst and optionally a ligand.

Ligands which optionally may be present include alcohols, sulphides and preferably amines, especially the substrate amines of formula (1). Preferred substrate amines of formula (1) are substituted amines of formula (1) wherein at least one of R¹⁴ is an optionally substituted hydrocarbyl comprising an α-methyl group.

When a ligand is used, optionally the ligand the transition metal catalyst may be pre-mixed or pre-coordinated prior to the reaction with the substrate. Examples of such pre-coordinated ligand and the transition metal catalysts include those catalysts disclosed in the International patent applications with publication numbers WO97/20789, WO98/42643, and WO02/44111, each of which is incorporated herein by reference, (>r catalysts such as bis-dicarbonyl[1-hydroxyl-2,3,4,5-tetraphenyl-cyclopentadienylruthenium(II)hydride described in Tet. Lett. 2002, 43, 4699; chlorodicarbonyl[1-(i-propylamino)-2,3,4,5-tetraphenylcyclopentadienylruthenium(II) described in J. Am. Chem. Soc. 2003, 125, 11494; and pincer complexes such as bis-1,3-ditertbutylphosphinomethyl-dihydroiridium-2-benzene described in J. Mol. Catal. A Chemical 189, 2002, 119.

Transition metal catalysts include transition metal halides, transition metal halide complexes and transition metal complexes wherein the transition metal is optionally complexed by a displaceable ligand.

Displaceable ligands include phosphines, such as tri-hydrocarbyl phosphines for example Ph₃P, carbenes such as imidazole carbene, nitrites such as acetonitrile, carbon monoxide, triflate, alkenes and dienes. Examples of transition metal complexes wherein the transition metal is optionally complexed by a displaceable ligand include complexes of the formula MₙLₒXₚYᵣ

Wherein
M is a transition metal;
L is a displaceable ligand;
X is a halide;
Y is a neutral optionally substituted hydrocarbyl complexing group, a neutral optionally substituted perhalogenated hydrocarbyl complexing group, or an optionally substituted cyclopentadienyl complexing group; and
n is an integer; and
each of o, p, and r is 0 or an integer provided that o+p+r is an integer.

Preferably, the transition metal catalyst is a transition metal halide or transition metal halide complex based on the transition metals in Group VIII of the Periodic Table, especially ruthenium, rhodium or iridium.

More preferably, the transition metal catalyst is a transition metal halide complex of the formula MₙXₚYᵣ

wherein
M is a transition metal;
X is a halide;
Y is a neutral optionally substituted hydrocarbyl complexing group, a neutral optionally substituted perhalogenated hydrocarbyl complexing group, or an optionally substituted cyclopentadienyl complexing group; and
n, p and r are integers.

Although transition metal catalyst is believed to be substantially as represented in the above formula, in some circumstances the transition metal catalyst may also exist as a dimer, trimer or some other polymeric species.

Halides which may be represented by X include chloride, bromide and iodide. Preferably, X is iodide.

Metals which may be represented by M include metals which are capable of catalysing transfer hydrogenation. Preferred metals include transition metals, more preferably the metals in Group VIII of the Periodic Table, (iron, cobalt, nickel, ruthenium, rhodium, palladium, osmium, iridium and platinum), more preferably ruthenium, rhodium or iridium, most preferably iridium.

Typically, the integers n, p, r are selected such that the transition metal halide complex is overall a neutral species. Therefore, the selection of n, p, r are directly related to the valance state of the metal and the number of halides present and the nature of the complexing group Y. For example, where Y is a negatively charged cyclopentadienyl complexing group, the number of negatively charged halides required to balance the valence state of the metal will be less than when Y is a neutral hydrocarbyl complexing group.

When the metal is ruthenium it is preferably present in valence state II. When the metal is rhodium or iridium it is preferably present in valence state I when Y is a neutral optionally substituted hydrocarbyl or a neutral optionally substituted perhalogenated hydrocarbyl ligand, and preferably present in valence state III when Y is an optionally substituted cyclopentadienyl ligand. An especially preferred metal is iridium.

The neutral optionally substituted hydrocarbyl or perhalogenated hydrocarbyl complexing group which may be represented by Y includes optionally substituted aryl and alkenyl complexing group.

Optionally substituted aryl complexing groups which may be represented by Y may contain 1 ring or 2 or more fused rings which include cycloalkyl, aryl or heterocyclic rings. Preferably, the complexing group comprises a 6 membered aromatic ring. The ring or rings of the aryl complexing group are often substituted with hydrocarbyl groups. The substitution pattern and the number of substituents will vary and may be influenced by the number of rings present, but often from 1 to 6 substituents are present. Substituents may include halogen, cyano, nitro, hydroxy, amino, thiol, acyl, hydrocarbyl, perhalogentated hydrocarbyl, heterocyclyl, hydrocarbyloxy, mono or di-hydrocarbylamino, hydrocarbylthio, esters, carbonates, amides, sulphonyl and sulphonamido groups wherein the hydrocarbyl groups are as defined for R¹ above. Typically, the 1 to 6 substituents are each independently hydrocarbyl groups, preferably 2, 3 or 6 hydrocarbyl groups and more preferably 6 hydrocarbyl groups. Preferred hydrocarbyl substituents include methyl, ethyl, iso-propyl, menthyl, neomenthyl and phenyl. Particularly when the aryl complexing group is a single ring, the complexing group is preferably benzene or a substituted benzene. When the complexing group is a perhalogenated hydrocarbyl, preferably it is a polyhalogenated benzene such as hexachlorobenzene or hexafluorobenzne. When the hydrocarbyl substitutents contain enantiomeric and/or diastereomeric centres, it is preferred that the enantiomerically and/or diastereomerically purified forms of these are used. Benzene, p-cymyl, mesitylene and hexamethylbenzene are especially preferred complexing group.

Optionally substituted alkenyl complexing groups which may be represented by Y include C₂₋₃₀, and preferably C₆₋₁₂, alkenes or cycloalkenes with preferably two or more carbon-carbon double bonds, preferably only two carbon-carbon double bonds. The carbon-carbon double bonds may optionally be conjugated to other unsaturated systems which may be present, but are preferably conjugated to each other. The alkenes or cycloalkenes may be substituted preferably with hydrocarbyl substituents. When the alkene has only one double bond, the optionally substituted alkenyl complexing group may comprise two separate alkenes. Preferred hydrocarbyl substituents include methyl, ethyl, iso-propyl and phenyl. Examples of optionally substituted alkenyl complexing groups include cyclo-octa-1,5-diene and 2,5-norbornadiene. Cyclo-octa-1,5-diene is especially preferred.

Optionally substituted cyclopentadienyl complexing groups which may be represented by Y includes cyclopentadienyl groups capable of eta-5 bonding. The cyclopentadienyl group is often substituted with from 1 to 5 substituents. Substituents may include halogen, cyano, nitro, hydroxy, amino, thiol, acyl, hydrocarbyl, perhalogentated hydrocarbyl, heterocyclyl, hydrocarbyloxy, mono or di-hydrocarbylamino, hydrocarbylthio, esters, carbonates, amides, sulphonyl and sulphonamido groups wherein the hydrocarbyl groups are as defined for R¹ above. Preferably, the cyclopentadienyl group is substituted with 1 to 5 hydrocarbyl groups, more preferably with 3 to 5 hydrocarbyl groups and most preferably with 5 hydrocarbyl groups. Preferred hydrocarbyl substituents include methyl, ethyl and phenyl. When the hydrocarbyl substitutents contain enantiomeric and/or diastereomeric centres, it may be advantageous that the enantiomerically and/or diastereomerically purified forms of these are used. Examples of optionally substituted cyclopentadienyl complexing groups include cyclopentadienyl, pentamethyl-cyclopentadienyl, pentaphenylcyclopentadienyl, tetraphenylcyclopentadienyl, ethyltetramethylpentaclienyl, menthyltetraphenylcyclopentadienyl, neomenthyltetraphenylcyclopentadienyl, menthylcyclopentadienyl, neomenthylcyclopentadienyl, tetrahydroindenyl, menthyltetrahydroindenyl and neomenthyltetrahydroindenyl groups. Pentamethylcyclopentadienyl is especially preferred.

Transition metal halide complexes of the formula MₙXₚYᵣ wherein M is Rh or Ir, and Y is an optionally substituted cyclopentadienyl group are preferred. Transition metal halide complexes of the formula MₙXₚYᵣ wherein M is Ir and Y is an optionally substituted cyclopentadienyl group are most preferred. Highly preferred are transition metal iodide complexes of the formula MₙIₚYᵣ, more preferably wherein M is Ir and Y is an optionally substituted cyclopentadienyl group.

Examples of transition metal halide complexes include Ru₂Cl₄(cymyl)₂, Rh₂Cl₄(Cp*)₂, Rh₂Br₄(Cp*)₂, Rh₂I₄(CP*)₂, Ir₂Cl₄(Cp*)₂, Ru₂I₄(Cymyl)₂, RhCl₂Cp*, RhBr₂Cp*, RhI₂ Cp*, and Ir₂I₄(Cp*)₂ wherein Cp is a pentamethylcyclopentadienyl group.

In certain preferred embodiments, the catalyst system is preferably a composition obtainable by contacting a transition metal halide complex of the formula MₙXₚYᵣ wherein M is a transition metal; X is a halide; Y is a neutral optionally substituted hydrocarbyl complexing group, a neutral optionally substituted perhalogenated hydrocarbyl complexing group, or an optionally substituted cyclopentadienyl complexing group; and n, p and r are integers with an amine ligand of formula (1).

The catalytic system may advantageously be introduced, at least in part, on a solid support or as an encapsulated system. Where the catalytic system is present on a solid support or as an encapsulated system, such supported catalyst systems may be of assistance in performing separation operations which may be required, and may facilitate the ease of cycling of materials between steps, especially when repetitions are envisaged. Examples of solid support or encapsulation technology that may be employed to support or encapsulate the catalytic system are described in WO03/006151 and WO05/016510.

Reaction promoters, which optionally may be present, include halide salts, for example metal halides. Preferred reaction promoters include bromide and especially iodide salts. Highly preferred are potassium iodide and caesium iodide.

In a further aspect of the present invention, the corresponding imines of formula (2)

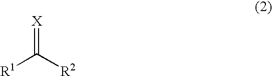

wherein:
X represents NR³, NR⁴, (NR³R⁴)⁺Q⁻;
Q⁻ represents an anion;
R¹, R² each independently represents an optionally substituted hydrocarbyl, a perhalogenated hydrocarbyl, an optionally substituted heterocyclyl group or a substitutent group;
R³ represents a hydrogen atom, an optionally substituted hydrocarbyl, a perhalogenated hydrocarbyl, an optionally substituted heterocyclyl group or a removable group;
R⁴ represents a hydrogen atom, an optionally substituted hydrocarbyl, a perhalogenated hydrocarbyl or an optionally substituted heterocyclyl group; or one or more of R¹ & R², R¹ & R³, R² & R⁴ and R³ & R⁴ optionally being linked in such a way as to form an optionally substituted ring(s),
   derived by dehydrogenation of the starting amines of formula (1) may be produced.

Advantageously, mines may be obtained under mild conditions without the need to use stoicheometric amounts of strong oxidants.

Where it is desired to suppress or promote the production of the corresponding imines derived by dehydrogenation of the starting amines of formula (1), the use of hydrogen acceptors and/or hydrogen donors may advantageously be employed.

Hydrogen acceptors which may be present in the process of the present invention include the proton from an acid, oxygen, aldehydes and ketones, imines and imminium salts, readily hydrogenatable hydrocarbons, dyes, clean oxidising agents, carbonates, bicarbonates and any combination thereof.

The proton may emanate from any convenient and compatible acid such as formic acid, acetic acid, hydrogen carbonate, hydrogen sulfate, ammonium salt or alkyl ammonium salt. Conveniently the proton may emanate from the substrate itself.

Aldehydes and ketones which may be employed as hydrogen acceptors comprise commonly from 1 to 20 carbon atoms, preferably from 2 to 15 carbon atoms, and more preferably 3 to 5 carbon atoms. Aldehydes and ketones include alkyl, aryl, heteroaryl aldehydes and ketones, and ketones with mixed alkyl, aryl or heteroaryl groups. Examples of aldehydes and ketones which may be represented as hydrogen acceptors include formaldehyde, acetone, methylethylketone and benzophenone. When the hydrogen donor is an aldehyde or ketone, acetone is especially preferred.

Readily hydrogenatable hydrocarbons which may be employed as hydrogen acceptors comprise hydrocarbons which have a propensity to accept hydrogen or hydrocarbons which have a propensity to form reduced systems. Examples of readily hydrogenatable hydrocarbons which may be employed by as hydrogen donors include quinones, dihydroarenes and tetrahydroarenes.

Clean oxidising agents which may be represented as hydrogen acceptors comprise reducing agents with a high reduction potential, particularly those having an oxidation potential relative to the standard hydrogen electrode of greater than about 0.1 eV, often greater than about 0.5 eV, and preferably greater than about 1 eV. Examples of clean oxidising agents which may be represented as hydrogen acceptors include oxidising metals and oxygen.

Dyes include Rose Bengal, Proflavin, Ethidium Bromide, Eosin and Phenolphthalein.

Carbonates and bicarbonates include alkali metal, alkaline earth metal, ammonium and quaternary amine salts of carbonate and bicarbonate.

The most preferred hydrogen acceptors are protons from acids, acetone, oxygen, the substrate amine and carbonate and bicarbonate salts.

Hydrogen donors include hydrogen, primary and secondary alcohols, primary secondary and tertiary amines, carboxylic acids and their esters and amine salts, readily dehydrogenatable hydrocarbons, clean reducing agents, and any combination thereof.

Primary and secondary alcohols which may be employed as hydrogen donors comprise commonly from 1 to 10 carbon atoms, preferably from 2 to 7 carbon atoms, and more preferably 3 or 4 carbon atoms. Examples of primary and secondary alcohols which may be represented as hydrogen donors include methanol, ethanol, propan-1-ol, propan-2-ol, butan-1-ol, butan-2-ol, cyclopentanol, cyclohexanol, benzylalcohol, and menthol. When the hydrogen donor is an alcohol, secondary alcohols are preferred, especially propan-2-ol and butan-2-ol.

Primary secondary and tertiary amines which may be employed as hydrogen donors comprise commonly from 1 to 20 carbon atoms, preferably from 2 to 14 carbon atoms, and more preferably 3 or 8 carbon atoms. Examples of primary, secondary and tertiary amines which may be represented as hydrogen donors include ethylamine, propylamine, isopropylamine, butylamine, isobutylamine, hexylamine, diethylamine, dipropylamine, di-isopropylamine, dibutylamine, di-isobutylamine, dihexylamine, benzylamine, dibenzylamine, piperidine, (R) or (S) 6,7-dimethoxy-1-methyldihydroisoquinoline, triethylamine. When the hydrogen donor is an amine, primary amines are preferred, especially primary amines comprising a secondary alkyl group, particularly isopropylamine and isobutylamine.

Carboxylic acids or their esters or salts which may be employed as hydrogen donors comprise commonly from 1 to 10 carbon atoms, preferably from 1 to 3 carbon atoms. In certain embodiments, the carboxylic acid is advantageously a beta-hydroxy-carboxylic acid. Esters may be derived from the carboxylic acid and a C₁₋₁₀ alcohol. Examples of carboxylic acids which may be employed as hydrogen donors include formic acid, lactic acid, ascorbic acid and mandelic acid. When a carboxylic acid is employed as hydrogen donor, at least some of the carboxylic acid is preferably present as a salt. Amine salts may be formed. Amines which may be used to form such salts include both aromatic and non-aromatic amines, also primary, secondary and tertiary amines and comprise typically from 1 to 20 carbon atoms. Tertiary amines, especially trialkylamines, are preferred. Examples of amines which may be used to form salts include trimethylamine, triethylamine, di-isopropylethylamine and pyridine. The most preferred amine is triethylamine. When at least some of the carboxylic acid is present as an amine salt, particularly when a mixture of formic acid and triethylamine is employed, the mole ratio of acid to amine is commonly about 5:2. This ratio may be maintained during the course of the reaction by the addition of either component, but usually by the addition of the carboxylic acid. Other preferred salts include sodium, potassium, magnesium

Readily dehydrogenatable hydrocarbons which may be employed as hydrogen donors comprise hydrocarbons which have a propensity to aromatise or hydrocarbons which have a propensity to form highly conjugated systems. Examples of readily dehydrogenatable hydrocarbons which may be employed by as hydrogen donors include cyclohexadiene, cyclohexene, tetralin, dihydrofuran and terpenes.

Clean reducing agents which may be represented as hydrogen donors comprise reducing agents with a high reduction potential, particularly those having a reduction potential relative to the standard hydrogen electrode of greater than about −0.1 eV, often greater than about −0.5 eV, and preferably greater than about −1 eV. Examples of clean reducing agents which may be represented as hydrogen donors include hydrazine and hydroxylamine.

The most preferred hydrogen donors are (R) or (S) 6,7-dimethoxy-1-methyldihydroisoquinoline, propan-2-ol, butan-2-ol, triethylammonium formate, sodium formate, potassium formate and a mixture of triethylammonium formate and formic acid.

Although gaseous hydrogen may be present, the process is normally operated in the absence of gaseous hydrogen since it appears to be unnecessary.

Typically, inert gas sparging may be employed.

Suitably the process is carried out at temperatures in the range of from minus 78 to plus 150° C., preferably from minus 20 to plus 110° C. and more preferably from plus 40 to plus 80° C.

The initial concentration of the substrate, a compound of formula (1), is suitably in the range 0.05 to 1.0 and, for convenient larger scale operation, can be for example up to 6.0 more especially 0.75 to 2.0, on a molar basis. The molar ratio of the substrate to the catalyst system is suitably no less than 50:1 and can be up to 50000:1, preferably between 250:1 and 5000:1 and more preferably between 500:1 and 2500:1.

If a reaction promoter is present, the reaction promoter is preferably employed in a molar excess over the substrate, especially from 1 to 5 fold or, if convenience permits, greater, for example up to 20 fold.

If a hydrogen donor and/or acceptor is present, the hydrogen donor and/or acceptor is preferably employed in a molar excess over the substrate, especially from 5 to 20 fold or, if convenience permits, greater, for example up to 500 fold.

Reaction times are typically in the range of from 1.0 min to 24 h, especially up to 8 h and conveniently about 3 h. After reaction, the mixture is worked up by standard procedures.

A reaction solvent may be present, for example dimethylformamide, acetonitrile, tetrahydrofuran, toluene, chloroform, dichloromethane or, conveniently, the substrate amine when the substrate amine is liquid at the reaction temperature. Preferred solvents include non-polar aromatic solvents such as toluene, mesitylene, p-cymene and cumene, and polar aprotic solvents such as dioxane, ethers, for example diethyl ether or tetrahydrofuran, and acetates, for example t-BuOAc. Usually it is preferred to operate in substantial absence of water, but water does not appear to unduly inhibit the reaction. When water is used as solvent, preferably a pH buffer is employed. When the substrate amine or the reaction solvent is not miscible with water and the desired product is water soluble, it may be desirable to have water present as a second phase. The concentration of substrate may be chosen to optimise reaction time, yield and de-enrichment of enantiomeric excess.

Advantageously, the process of the present invention may find use in recycling unwanted isomers obtained from chiral processes, such as chiral separations, chemical and enzymic chiral resolutions and the likes. Typically, in chiral separations or resolutions, racemic mixtures are subjected to physical, chemical or biochemical treatments which result in the separation of a desired enantiomer or enantiomeric product while often leaving behind an unreacted or unwanted enantiomers or enantiomeric bi-products. The process of the present invention provides a method for converting the unreacted enantiomers to usable feedstocks containing wanted enantiomers.

The invention is illustrated by the following Examples.

### EXAMPLE 1

### Racemisation and Dehydrogenation (S) 6,7-dimethoxy-1-methyl-1,2,3,4-tetrahydroisoquinoline in Chloroform/Tetrahydrofuran at 40° C.

Into a 5 ml round bottom flask was added pentamethylcyclopentadienyliridium(III)chloride dimer (8.30 mg 96%, 7.97 mg=0.01 mmol). Chloroform (250 μl) was added and the catalyst solution was stirred using a magnetic stirrer until all the catalyst had dissolved resulting in an orange solution. (S)-6,7-dimethoxy-1-methyl-1,2,3,4-tetrahydroisoquinoline (218.2 mg 95%, 207.25 mg=1.00 mmol) and potassium iodide (167.7 mg 99%, 166.01 mg=1.00 mmol) were added and washed in using tetrahydrofuran (750 μl). The flask was fitted with a water condenser, and then placed in an oil bath at 40° C., and a timer was started.

The potassium iodide remained predominantly out of solution, after about five mins at 40° C. the reaction solution had become brown and remained this colour throughout.

Samples were taken at regular intervals by adding 40 μl of the reaction solution to dichloromethane (2 ml) and 2.5M sodium hydroxide solution (2 ml). The organic layer was separated and dried using sodium sulphate. The resulting solution was analysed by chiral and achiral g.c.

### Analysis

### Gas Chromatography (for Conversion)

Varian CP-SIL 8CB column (25 m, 320 μm, 0.12 μm), 150° C. isothermal, 12.0 psi, 10 mins. 25° C./min for 4 mins then 4 mins at 250° C.

Amine=4.8-5.0 mins.
Imine=5.19-5.21 mins.

### Gas Chromatography (for ee)

Varian Chirasil -Dex-CB column (25 m, 250 μm, 0.25 μm), 165° C. isothermal for 60 mins.

N.B. One drop of trifluoroacetic anhydride was added to each sample vial prior to injection.
Enantiomer 1 retention time=46.3-46.5 mins
Enantiomer 2 retention time=47.2-47.4 mins

### Results

| Time/ | | | | % Amine | % Amine |
|---|---|---|---|---|---|
| mins | % Amine | % Imine | % ee | Enantiomer 1 | Enantiomer 2 |
| 0 | 96.4 | 3.6 | 98.8 | 0.6 | 99.4 |
| 10 | 91.8 | 8.2 | 30.2 | 34.9 | 65.1 |
| 30 | 86.5 | 12.7 | 8.5 | 45.75 | 54.25 |
| 65 | 82.9 | 16.2 | 2.5 | 48.75 | 51.25 |
| 120 | 66.9 | 30.5 | 2.5 | 48.75 | 51.25 |

### EXAMPLE 2

A solution of pentamethylcyclopentadienyliridium(III) chloride dimer was prepared by dissolving the dimer (16.6 mg 96%, 15.9 mg, 0.020 mmol) in chloroform (0.5 ml) resulting in a dark orange solution (Iridium Catalyst solution).

A solution of iodine was prepared by dissolving the solid (17.1 mg 99%, 16.9 mg, 0.067 mmol) in tetrahydrofuran (0.5 ml) this resulted in a dark brown solution (Iodine solution) (75 μl of this solution corresponds to 0.01 mmol iodine).

A small quantity of the potassium iodide used for these reactions was placed in the oven at 170° C. and dried to constant weight. A second sample of the potassium iodide was ground to a fine powder then placed in the oven and dried to constant weight (˜1% weight loss).

Seven G.C. vials were set up containing a magnetic flea and (R)-N-Methyl-α-methylbenzylamine (13.8 mg 98%, 13.5 mg, 0.10 mmol) the following material was added to each vial:—
1. Caesium iodide (26.0 mg 99.9%, 26.0 mg, 0.10 mmol), tetrahydrofuran (75 μl) and the iridium catalyst solution (25 μl).
2. Potassium iodide (15.1 mg 99%, 14.9 mg, 0.10 mmol), iodine solution (75 μl) and the iridium catalyst solution (25 μl).
3. Iodine solution (75 μl) and the iridium catalyst solution (25 μl).
4. Potassium bromide (12.0 mg 99%, 11.9 mg, 0.09 mmol), iodine solution (75 μl) and the iridium catalyst solution (25 μl).
5. Potassium iodide dried powder (16.8 mg 99%, 16.6 mg, 0.10 mmol), iodine solution (75 μl) and the iridium catalyst solution (25 μl).
6. Potassium iodide (16.8 mg 99%, 16.6 mg, 0.10 mmol), iodine solution (75 μl) and the iridium catalyst solution (25 μl).
7. Dried potassium iodide (16.8 mg 99%, 16.6 mg, 0.10 mmol), iodine solution (75 μl) and the iridium catalyst solution (25 μl).

Each vial was sealed and placed in a stem block at 40° C., samples were taken after stirring overnight by quenching 20 μl of the reaction solution into dichloromethane (2 ml) and 2.5 M sodium hydroxide (2 ml), the organic layer was separated, dried using sodium sulphate and analysed by chiral and achiral G.C.

### Analysis

### Gas Chromatography (for Conversion)

HP-5 5% Methyl Phenyl Siloxane capillary column (30 m, 320 μm, 0.25 μm), 70° C. isothermal for 30 mins, 15.0 psi.
N-methyl-α-methylbenzylamine=6.8 mins

### Gas Chromatography (for ee)

Varian Chirasil -Dex-CB column (25 m, 250 m, 0.25 m), 100° C. isothermal for 60 mins. N.B. One drop of trifluoroacetic anhydride was added to each sample vial prior to injection.
R enantiomer N-methyl-α-methylbenzylamine=58.4 mins
S enantiomer N-methyl-α-methylbenzylamine=54.8 mins

| Experiment | Description | % Amine | % ee |
|---|---|---|---|
| 1 | +CsI | 97.0 | 72.5 |
| 2 | +0.1 eq I₂ + 0.9 eq KI | 99.0 | 98.2 |
| 3 | +0.1 eq I₂ | 98.7 | 98.6 |
| 4 | +KBr + 0.1 eq I₂ | 98.8 | 98.2 |
| 5 | +Dried KI powder | 94.8 | 79.5 |
| 6 | +KI | 95.3 | 81.1 |
| 7 | +Dried KI | 94.5 | 79.7 |

### EXAMPLE 3

### Gas Purges

### Ex. 3.1

### Air Purge

Pentamethylcyclopentadienyliridium(III) chloride dimer (16.6 mg 96%, 15.9 mg, 0.02 mmol), (R)-N-methyl-α-methylbenzylamine (275.9 mg 98%, 270.4 mg, 2.00 mmol) and potassium iodide (335.4 mg 99%, 332.0 mg, 2.00 mmol) were charged to a 5 ml round-bottom flask. Toluene (4 ml) was added and a water condenser was attached to the flask which was then placed in a oil bath at 80° C. An air purge was passed through the reaction solution (10 ml/min) and a timer was started. The reaction solution immediately turned to a dark red/brown that faded over 60 mins to a dark orange solution. The colour of the solution gradually faded and was a clear orange solution after stirring overnight.

In order to replace solvent lost due to the purge, toluene (2 ml) was added after five hours.

Samples were taken at regular intervals by removing 100-200 μl and quenching into dichloromethane (2 ml) and 2.5 M sodium hydroxide (2 ml), the organic layer was separated and dried using sodium sulphate. The resulting organic layer was analysed by chiral and achiral g.c.

### Ex. 3.2

### No Purge

Pentamethylcyclopentadienyliridium(III) chloride dimer (16.6 mg 96%, 15.9 mg, 0.02 mmol), (R)-N-methyl-α-methylbenzylamine (275.9 mg 98%, 270.4 mg, 2.00 mmol), potassium iodide (335.4 mg 99%, 332.0 mg, 2.00 mmol) and biphenyl (155.0 mg 99.5%, 154.2 mg, 1.00 mmol) were charged to a 5 ml round-bottom flask. Toluene (4 ml) was added, a water condenser was attached to the flask, the flask was then placed in an oil bath at 80° C. and a timer was started. The reaction solution immediately turned to a dark red/brown that faded over 60 mins to a dark orange solution. The colour of the solution gradually faded and was a clear orange solution after stirring overnight.

Samples were taken at regular intervals by removing 100-200 μl and quenching into dichloromethane (2 ml) and 2.5 M sodium hydroxide (2 ml), the organic layer was separated and dried using sodium sulphate. The resulting organic layer was analysed by chiral and achiral g.c.

### Ex. 3.3

### Nitrogen Purge

Pentamethylcyclopentadienyliridium(III) chloride dimer (16.6 mg 96%, 15.9 mg, 0.02 mmol), (R)-N-methyl-α-methylbenzylamine (275.9 mg 98%, 270.4 mg, 2.00 mmol), potassium iodide (335.4 mg 99%, 332.0 mg, 2.00 mmol) and n-decane (143.4 mg 99%, 142.0 mg, 1.00 mmol) were charged to a 5 ml round-bottom flask. Toluene was degassed by sparging through the solvent with nitrogen for 30 minutes then 4 ml was added and a water condenser was attached to the flask which was then placed in a oil bath at 80° C. and a timer was started. A nitrogen purge was passed through the reaction solution (10 ml/min). The reaction solution immediately turned to a dark red/brown that faded over 60 mins to a dark orange solution. The colour of the solution gradually faded and was a clear orange solution after stirring overnight. In order to replace solvent lost due to the purge toluene (2 ml) was added after 325 minutes.

Samples were taken at regular intervals by removing 100-200 μl and quenching into dichloromethane (2 ml) and 2.5 M sodium hydroxide (2 ml), the organic layer was separated and dried using sodium sulphate. The resulting organic layer was analysed by chiral and achiral g.c.

### Analysis

### Gas Chromatography (for Conversion)

HP-5 5% Methyl Phenyl Siloxane capillary column (30 m, 320 μm, 0.25 μm), 70° C. isothermal for 30 mins, 15.0 psi.
N-methyl-α-methylbenzylamine=6.8 mins

### Gas Chromatography (for ee)

### N-methyl-α-methylbenzylamine

Varian Chirasil -Dex-CB column (25 m, 250 μm, 0.25 μm), 100° C. isothermal for 60 mins. N.B. One drop of trifluoroacetic anhydride was added to each sample vial prior to injection.
R enantiomer N-methyl-α-methylbenzylamine=58.4 mins
S enantiomer N-methyl-α-methylbenzylamine=54.8 mins
Racemisation of (R)-N-Methyl-α-methylbenzylamine in Toluene at 80° C. Using [IrCp*Cl₂]₂+KI+Air Purge.

| Time | % Amine | % ee |
|---|---|---|
| 0 | 100.0 | 100.0 |
| 60 | 99.8 | 32.7 |
| 180 | 99.5 | 8.5 |
| 280 | 99.2 | 4.2 |
| 1245 | 97.4 | 0.2 |

Racemisation of (R)-N-Methyl-α-methylbenzylamine in Toluene at 80° C. Using [IrCp*Cl₂]₂+KI (without a Pure).

| Time | % Amine | % ee |
|---|---|---|
| 0 | 100.0 | 98.9 |
| 30 | 99.4 | 69.2 |
| 60 | 100.0 | 49.8 |
| 90 | 100.0 | 37.3 |
| 120 | 100.0 | 30.4 |
| 180 | 100.0 | 25.1 |
| 240 | 98.4 | 23.1 |
| 300 | 98.1 | 20.8 |
| 1320 | 96.2 | 6.3 |

Racemisation of (R)-N-Methyl-α-methylbenzylamine in Toluene at 80° C. Using [IrCp*Cl₂]₂+KI+Nitrogen Purge.

| Time | % Amine | % ee | R-amine (moldm⁻³) | S-amine (mold ⁻³) |
|---|---|---|---|---|
| 0 | 100.0 | 98.7 | 0.49675 | 0.00325 |
| 30 | 100.0 | 59.8 | 0.3995 | 0.1005 |
| 60 | 100.0 | 33.1 | 0.33275 | 0.16725 |
| 95 | 100.0 | 18.4 | 0.296 | 0.204 |
| 120 | 100.0 | 12.3 | 0.28075 | 0.21925 |
| 180 | 100.0 | 4.6 | 0.615 | 0.2385 |
| 240 | 100.0 | 2.5 | 0.25625 | 0.24375 |
| 325 | 100.0 | 1.0 | 0.2525 | 0.2475 |
| 1440 | 97.8 | 0.0 | 0.25 | 0.25 |

### EXAMPLE 4

Pentamethylcyclopentadienyliridium(III) chloride dimer (16.6 mg 96%, 15.9 mg, 0.02 mmol), (R)-N-methyl-α-methylbenzylamine (275.9 mg 98%, 270.4 mg, 2.00 mmol), potassium iodide (335.4 mg 99%, 332.0 mg, 2.00 mmol) and tridecane (186.2 mg 99%, 184.4 mg, 1.00 mmol) were charged to a 5 ml round-bottom flask. Toluene (4 ml) was added, a water condenser was attached to the flask, the flask was then placed in an oil bath at 80° C., and a timer was started. The reaction solution immediately turned to a dark red/brown that faded over 60 mins to a dark orange solution. The colour of the solution gradually faded and was a clear orange solution after stirring overnight.

Samples were taken at regular intervals by removing 100-200 μl and quenching into dichloromethane (2 ml) and 2.5 M sodium hydroxide (2 ml), the organic layer was separated and dried using sodium sulphate. The resulting organic layer was analysed by chiral and achiral g.c.

### Analysis

### Gas Chromatography (for Conversion)

HP-5 5% Methyl Phenyl Siloxane capillary column (30 m, 320 μm, 0.25 μm), 70° C. isothermal for 30 mins, 15.0 psi.
N-methyl-α-methylbenzylamine=6.8 mins

### Gas Chromatography (for ee)

### N-methyl-α-methylbenzylamine

Varian Chirasil -Dex-CB column (25 m, 250 μm, 0.25 μm), 100° C. isothermal for 60 mins. N.B. One drop of trifluoroacetic anhydride was added to each sample vial prior to injection.
R enantiomer N-methyl-α-methylbenzylamine=58.4 mins
S enantiomer N-methyl-α-methylbenzylamine=54.8 mins
Racemisation of (S)-N-Methyl-α-methylbenzylamine in Toluene at 80° C. using [IrCp*Cl₂]₂+KI (No Purge Used)

| | | | R amine | S amine |
|---|---|---|---|---|
| Time | % Amine | % ee | (moldm⁻³) | (moldm⁻³) |
| 0 | 99.6 | 100.0 | 0 | 0.5 |
| 30 | 99.3 | 57.6 | 0.106 | 0.394 |
| 60 | 99.1 | 34.5 | 0.163 | 0.336 |
| 95 | 98.9 | 22.4 | 0.194 | 0.306 |
| 122 | 98.8 | 17.8 | 0.206 | 0.294 |
| 180 | 98.3 | 14.9 | 0.213 | 0.287 |
| 240 | 98.3 | 12.6 | 0.218 | 0.282 |
| 300 | 98.0 | 11.9 | 0.22 | 0.28 |
| 1260 | 96.8 | 2.2 | 0.245 | 0.256 |

### EXAMPLE 5

Pentamethylcyclopentadienyliridium(III) iodide dimer (24.2 mg 96%, 23.25 mg, 0.02 mmol), (S)-N-methyl-α-methylbenzylamine (275.9 mg 98%, 270.4 mg, 2.00 mmol) and tridecane (186.2 mg 99%, 184.4 mg, 1.00 mmol) were charged to a 5 ml round-bottom flask. Toluene (4 ml) was added and a water condenser was attached to the flask which was then placed in a oil bath at 80° C. and a timer was started. The reaction solution immediately turned to a dark red/brown that faded over 60 mins to a dark orange solution. The colour of the solution gradually faded and was a clear orange solution after stirring overnight.

Samples were taken at regular intervals by removing 100-200 μl and quenching into dichloromethane (2 ml) and 2.5 M sodium hydroxide (2 ml), the organic layer was separated and dried using sodium sulphate. The resulting organic layer was analysed by chiral and achiral g.c.

### Analysis

### Gas Chromatography (for Conversion)

HP-5 5% Methyl Phenyl Siloxane capillary column (30 m, 320 μm, 0.25 μm), 70° C. isothermal for 30 mins, 15.0 psi.
N-methyl-α-methylbenzylamine=6.8 mins

### Gas Chromatography (for ee)

### N-methyl-α-methylbenzylamine

Varian Chirasil -Dex-CB column (25 m, 250 μm, 0.25 μm), 100° C. isothermal for 60 mins. N.B. One drop of trifluoroacetic anhydride was added to each sample vial prior to injection.
R enantiomer N-methyl-α-methylbenzylamine=58.4 mins
S enantiomer N-methyl-α-methylbenzylamine=54.8 mins

| | | | | R amine | S amine |
|---|---|---|---|---|---|
| Time | % Amine | % Impurities | % ee | (moldm⁻³) | (moldm⁻³) |
| 0 | 98.2 | 1.8 | 100.0 | 0 | 0.5 |
| 15 | 98.3 | 1.7 | 79.6 | 0.051 | 0.449 |
| 30 | 98.2 | 1.8 | 62.5 | 0.09375 | 0.40625 |
| 60 | 97.6 | 2.4 | 40.0 | 0.15 | 0.35 |
| 150 | 94.9 | 5.1 | 22.7 | 0.19325 | 0.30675 |
| 180 | 96.0 | 4.0 | 21.1 | 0.19725 | 0.30275 |
| 240 | 95.8 | 4.2 | 19.4 | 0.2015 | 0.2985 |
| 452 | 95.6 | 4.4 | 14.8 | 0.213 | 0.287 |
| 1322 | 93.7 | 6.3 | 4.6 | 0.2385 | 0.2615 |

### Preparation of Pentamethylcyclopentadienyliridium(III)iodide Dimer

Pentamethylcyclopentadienyliridium(III)chloride dimer (265.5 mg 96%, 254.9 mg, 0.32 mmol) and sodium iodide (499.6 mg 99%, 494.6 mg, 3.30 mmol) were added to a 3-neck 50 ml round bottom flask. A water condenser was fitted to the flask, the remaining necks were stoppered and argon was sparged through the vessel at 50 ml/min for 30 minutes. The purge of argon was then reduced to 5 ml/min and anhydrous acetone (30 ml) was added, the reaction flask was then placed in an oil bath at 60° C. and stirred using a magnetic stirrer resulting in a dark orange solution containing some insoluble iridium dimer. The reaction was allowed to reflux under argon for 3 hours before being cooled to room temperature. T.L.C. of the reaction solution (90% dichloromethane 10% methanol) indicated that the reaction had gone completely to a single new compound. The reaction was concentrated to dryness under vacuum to yield a brown/red solid that was dissolved in dichloromethane (50 ml) and washed with ultra pure water (2×25 ml), the organic layer was separated, dried using sodium sulphate, filtered and concentrated to dryness under vacuum to yield a brown solid (370.9 mg). The solid was recrystallised from chloroform/methanol to yield 183.9 mg of brown needle like crystals (49.4% yield).

The crystals were analysed by carbon and proton n.m.r. and for carbon/hydrogen ratio.

### Analysis

Elemental analysis

| | | |
|---|---|---|
| Calculated:- | | C = 20.66%, H = 2.60%, N = 0.00% |
| Found:- | 1st Run: | C = 20.90%, H = 2.51%, N = 0.13%. |
| | 2nd Run: | C = 20.86%, H = 2.44%, N = 0.00% |

n.m.r.
Cp* protons=1.83 ppm singlet
Cp* quaternary carbons=89.3 ppm
Cp* methyl carbons=11.13 ppm

### Repeat Preparation at Larger Scale

Pentamethylcyclopentadienyliridium(III)chloride dimer (4.57 96%, 4.38 g, 5.507 mmol) and sodium iodide (8.55 g 99%, 8.46 g, 56.7 mmol) were added to a single neck 1000 ml round bottom flask. A water condenser was fitted to the flask, the remaining necks were stoppered and argon was sparged through the vessel at 500 ml/min for 30 minutes. The purge of argon was then reduced to 20 ml/min and anhydrous acetone (525 ml) was added, the reaction flask was then placed in an oil bath at 60° C. and stirred using a magnetic stirrer resulting in a dark orange solution containing some insoluble iridium dimer. The reaction was allowed to reflux under argon for 3 hours before being cooled to room temperature. The reaction was concentrated to dryness under vacuum to yield a brown/red solid that was dissolved in dichloromethane (500 ml) and washed with ultra pure water (3×250 ml), the organic layer was separated, dried using sodium sulphate, filtered and concentrated to dryness under vacuum to yield a brown solid. The solid was recrystallised from chloroform/methanol to yield brown needle like crystals, the filtrates were concentrated to dryness and the resulting residue was recrystallised from chloroform/methanol, this was repeated a third time and the three crops of catalyst combined to yield 5.102 g (78.2% yield).

General Procedure for Amine Racemisation (2 mmol Scale)

To a 5 ml single-neck round-bottom flask is added the chiral amine (2 mmol), pentamethylcyclopentadienyl iridium (III) iodide dimer* (0.02 mmol) and toluene (4 ml). A condenser is attached to the flask and it is placed into an oil bath at 80° C. and agitated using a magnetic stirrer. *An alternative procedure involves the addition of pentamethylcyclopantadienyl iridium (III) chloride dimer (0.02 mmol) and potassium iodide (2 mmol).

Samples are taken at regular intervals and analysed for conversion and enantiomeric excess.

| | | | Time |
|---|---|---|---|
| | | Enantiomeric | taken to |
| | | excess after | reach |
| Exam- | | stirring for | <10% ee/ |
| ples | Amine | 16 h (%) | mins |
| 6 | 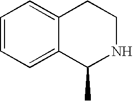 | 0% | 100 mins |
| 7 | 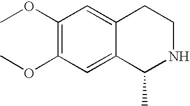 | 9% | 180 mins |
| 8 | 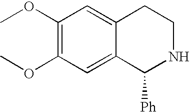 | 0% | 100 mins |
| 9 | 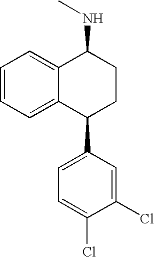 | 15% D.E. | Not applicable |
| 10 | 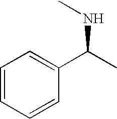 | 4% | ~800 mins |
| 11 | 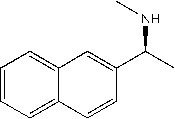 | 44.5% | Not applicable |
| 12 | 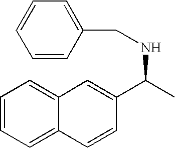 | 62% | Not applicable |
| 13 | 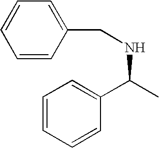 | 2% | ~900 mins |
| 14 | 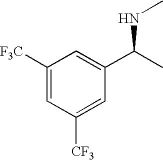 | 92% | Not applicable |
| 15 | 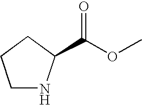 | 90% | Not applicable |
| 16 | 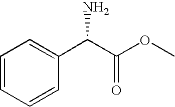 | 88% | Not applicable |
| 17 | 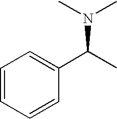 | 51% | ~3750 mins |
| 18 | 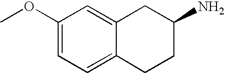 | 18% | Not applicable |

### EXAMPLE 19

### Racemisation of a Tertiary Amine in a Variety of Solvents

To 5 small vials was added (S)-(−)-N,N-dimethyl-1-phenethylamine (100 mg, 0.67 mmol), pentamethylcyclopentadienyliridium (III) iodide dimer (7.8 mg, 0.0067 mmol) in the following solvents: Toluene, tert-butylacetate, cyclopentylmethyl ether and diisopropyl alcohol (3 ml). A water condenser was attached and the reaction vessels heated to 90° C. Samples were taken at regular intervals (ca. 100 μl) and quenched into DCM (3 ml), and analysed by chiral G. C.

### Analytical Method

### Chiral G. C.

Method—steve55
CP chirasil dex CB DF=0.25 25 m×0.25 mm
Film thickness=0.25 μm

### Pressure=25.0 psi

Flow=3.2 ml/min
Temperature=55° C. isothermal for 80 minutes.
(S)-(−)-N,N-dimethyl-1-phenethylamine=73 mins
(R)-(+)-N,N-dimethyl-1-phenethylamine=71 mins

| Time | S | R | S | R | S | R | S | R |
|---|---|---|---|---|---|---|---|---|
| (min) | (Toluene) | (Toluene) | (tBuAc) | (tBuAc) | (CPME) | (CPME) | (DIPA) | (DIPA) |
| 10 | 99 | 1 | 100 | 0 | 100 | 0 | 100 | 0 |
| 30 | 98 | 2 | 99 | 1 | 100 | 0 | 100 | 0 |
| 60 | 96 | 4 | 98 | 2 | 98 | 2 | 98 | 2 |
| 120 | 88 | 12 | 95 | 5 | 96 | 4 | 96 | 4 |
| 240 | 89 | 11 | 94 | 6 | 93 | 7 | 92 | 8 |
| 600 | 78 | 23 | 87 | 13 | 86 | 14 | 85 | 5 |

### EXAMPLE 20

### Racemisation of a Tertiary Amine in Two Phase Systems

To four separate 10 ml round bottom flasks was added (S)-(−)-N,N-dimethyl-1-phenethylamine (100 mg, 0.67 mmol), pentamethylcyclopentadienyliridium (III) iodide dimer (7.8 mg, 0.0067 mmol) in a) toluene/water 1/1 (3 ml) and b) toluene/pH 7 buffer 1/1 (3 ml). A water condenser was attached and the reaction vessels heated to 90° C. Samples were taken at regular intervals (ca. 100 μl) and quenched into DCM/NaOH 2M (3 ml), extracted and dried (MgSO₂) and analyzed by chiral G. C.

### Analytical Method

### Chiral G. C.

Method—steve55
CP chirasil dex CB DF=0.25 25 m×0.25 mm
Film thickness=0.25 μm

### Pressure=25.0 psi

Flow=3.2 ml/min
Temperature=55° C. isothermal for 80 minutes.
(S)-(−)-N,N-dimethyl-1-phenethylamine=73 mins
(R)-(+)-N,N-dimethyl-1-phenethylamine=71 mins

| | S | S |
|---|---|---|
| Time | (tol/pH 7) | (tol/H2O) |
| 10 | 100 | 100 |
| 30 | 100 | 100 |
| 60 | 100 | 100 |
| 120 | 97 | 97 |
| 240 | 94 | 95 |
| 600 | 90 | 91 |

1. A process for the de-enrichment of enantiomerically enriched compositions comprising reacting an enantiomerically enriched composition comprising at least a first enantiomer or diastereomer of a substrate comprising a carbon-heteroatom bond, wherein the carbon is a chiral center and the heteroatom is a group V heteroatom, in the presence of a catalyst system and optionally a reaction promoter to give a product composition comprising first and second enantiomers or diastereomers of the substrate having a carbon-heteroatom bond, the ratio of second to first enantiomer or diastereomer in the product composition being greater than the ratio of second to first enantiomer or diastereomer in the enantiomerically enriched composition.

2. The process of claim 1, wherein the substrate comprising the carbon-heteroatom bond, the carbon atom being a chiral center, is a compound of formula (1): 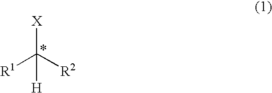
wherein:
X is NHR³, NR³R³, (NHR³R⁴)⁺Q⁻;
Q⁻ is an anion;
R¹ and R² each independently an optionally substituted hydrocarbyl, a perhalogenated hydrocarbyl, an optionally substituted heterocyclyl group or a substituent group;
R³ is a hydrogen atom, an optionally substituted hydrocarbyl, a perhalogenated hydrocarbyl, an optionally substituted heterocyclyl group or a removable group;
R⁴ is a hydrogen atom, an optionally substituted hydrocarbyl, a perhalogenated hydrocarbyl or an optionally substituted heterocyclyl group; or one or more of R¹ and R², R¹ and R³, R² and R⁴ and R³ & R⁴ form an optionally substituted ring(s);
provided that R¹, R², R³ and R⁴ are selected such that * is a chiral center.

3. The process of claim 1, wherein the catalyst system comprises a transition metal catalyst and optionally a ligand.

4. The process of claim 3, wherein the transition metal catalyst is a transition metal halide complex of the formula MₙXₚYᵣ
wherein
M is a transition metal;
X is a halide;
Y is a neutral optionally substituted hydrocarbyl complexing group, a neutral optionally substituted perhalogenated hydrocarbyl complexing group, or an optionally substituted cyclopentadienyl complexing group; and
n, p and r are integers.

5. The process of claim 4, wherein X is I.

6. The process of claim 4, wherein M is Rh or Ir, and Y is an optionally substituted cyclopentadienyl group.

7. The process of claim 6, wherein M is Ir, X is I, and Y is an optionally substituted cyclopentadienyl group.

8. The process of claim 6, wherein the transition metal catalyst is a transition metal halide complex of the formula M₂X₄Y₂ wherein M is Ir, X is I, and Y is an optionally substituted cyclopentadienyl group.

9. The process of claim 1, wherein a reaction promoter is present.

10. The process of claim 9, wherein the reaction promoter is a halide salt.

11. The process of claim 10, wherein the halide salt is a metal halide.

12. The process of claim 11, wherein the metal halide is potassium or cesium iodide.

13. The process of claim 1, wherein a compound of formula (2): 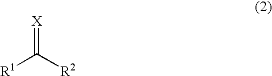
wherein:
X is NR³, NR⁴, (NR³R⁴)⁺Q⁻;
Q⁻ is an anion;
R¹ and R² each independently an optionally substituted hydrocarbyl, a perhalogenated hydrocarbyl, an optionally substituted heterocyclyl group or a substitutent group;
R³ is a hydrogen atom, an optionally substituted hydrocarbyl, a perhalogenated hydrocarbyl, an optionally substituted heterocyclyl group or a removable group;
R⁴ is a hydrogen atom, an optionally substituted hydrocarbyl, a perhalogenated hydrocarbyl or an optionally substituted heterocyclyl group, or
one or more of R¹ and R², R¹ and R³, R² and R⁴ and R³ and R⁴ form an optionally substituted ring(s), is obtained.

14. The process of claim 1, wherein a hydrogen donor or hydrogen acceptor is present.

15. The process of claim 1, wherein the enantiomerically enriched composition comprising at least a first enantiomer or diastereomer of a substrate comprising a carbon-heteroatom bond is an unreacted enantiomer or bi-product obtained from a chiral separation, or chemical or enzymatic chiral resolution.

16. A composition obtained by contacting a transition metal halide complex of the formula MₙXₚYᵣ wherein M is a transition metal; X is a halide; Y is a neutral optionally substituted hydrocarbyl complexing group, a neutral optionally substituted perhalogenated hydrocarbyl complexing group, or an optionally substituted cyclopentadienyl complexing group; and n, p and r are integers with an amine ligand of formula (1) 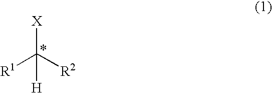
wherein:
X is NHR³, NR³R⁴, (NHR³R⁴)⁺Q⁻;
Q⁻ is an anion;
R¹ and R² each independently an optionally substituted hydrocarbyl, a perhalogenated hydrocarbyl, an optionally substituted heterocyclyl group or a substitutent group;
R³ is a hydrogen atom, an optionally substituted hydrocarbyl, a perhalogenated hydrocarbyl, an optionally substituted heterocyclyl group or a removable group;
R⁴ is a hydrogen atom, an optionally substituted hydrocarbyl, a perhalogenated hydrocarbyl or an optionally substituted heterocyclyl group; or one or more of R¹ and R², R¹ and R³, R² and R⁴ and R³ and R⁴ form an optionally substituted ring(s);
provided that R¹, R², R³ and R⁴ are selected such that * is a chiral center.
